(19) United States
(12) Patent Application Publication
(10) Pub. No.: US 2022/0401647 A1
(43) Pub. Date: Dec. 22, 2022

(54) HEMODIALYSIS SYSTEM INCLUDING CONTINUOUS GLUCOSE MONITORING

(71) Applicant: Bellco SRL, Mirandola (IT)

(72) Inventor: Roberto Carlos Chachero Gonzalez, Almazcara (ES)

(73) Assignee: Bellco SRL, Mirandola (IT)

(21) Appl. No.: 17/349,978

(22) Filed: Jun. 17, 2021

(51) Int. Cl. A61M 5/172; A61M 1/16; A61B 5/00; A61B 5/145; A61B 5/1455; A61B 5/1468

(52) U.S. Cl. CPC A61M 5/1723; A61M 1/1621; A61M 1/1601; A61B 5/0075; A61B 5/14532; A61B 5/1455; A61B 5/1468; A61B 5/6846; A61B 5/6801; A61M 2230/201; A61M 2205/18

(57) ABSTRACT

Systems and method for monitoring the blood glucose concentration of a patient during a hemodialysis session and automated administration of a medication in response to the glucose concentration falling outside a specified range. The system includes a hemodialysis system and a glucose sensor. The hemodialysis system includes a control system, at least one medication infusion pump, and a dialyzer fluidly connectable to a venous patient line and an arterial patient line. The glucose sensor is in communication with the control system and positioned to continuously measure the blood glucose concentration of the patient during the hemodialysis session. The control system can be programmed to provide automated administration of medication by the at least one medication infusion pump in response to changes in the blood glucose concentration of the patent during the hemodialysis session.

### FIELD

Systems, methods, and devices for continuous glucose monitoring and treatment of a patient during hemodialysis treatment are provided.

### BACKGROUND

The mortality rate of ESRD patients who receive traditional hemodialysis therapy is 23% per year with an even higher mortality rate among diabetic patients. For example, a patient with diabetes mellitus and established renal involvement (i.e., with albuminuria and decreased glomerular filtering) has a mortality rate, at ten years, up to 10 times higher than a diabetic patient without kidney disease. Diabetic patients also have significant differences with other hemodialysis patients in their demographic characteristics, complications, comorbidities, and treatment goals. A hemodialysis patient having diabetes needs special management in most areas of hemodialysis, such as hemodialysis guidelines, vascular access or diabetes control, etc. In many cases diabetic patients have significant variations in blood glucose figures during hemodialysis sessions, both hyperglycemia and symptomatic hypoglycemia, and other subsidiaries of immediate treatment.

However, the available systems and methods fail to monitor diabetic patients throughout a hemodialysis session. The available systems and methods commonly fail to ensure patient stability and cannot automatically predict possible alterations and incidences of hypoglycemia and hyperglycemia of the patient's glucose during a hemodialysis session. Although the available hemodialysis systems and methods attempt to monitor oxygen saturation, hematocrit and blood temperature, the systems and methods do not provide for the simultaneous and continuous monitoring of blood glucose during a hemodialysis session. Furthermore, these known systems commonly fail to include a means for treating the instance of hypoglycemia or hyperglycemia in the first place. The available systems and methods do not use sensors that can monitor and/or control glucose levels. As a result, healthcare professionals and clinicians are required to perform manual controls during hemodialysis to verify a patient's stability. The extra steps impose added economic cost, the use of consumables such as glucose strips, and require more time.

Hence, there is a need for systems and methods that provide continuous monitoring of a glucose concentration of a patient. The need can include both diabetic and non-diabetic patients. There is a further need for automating the administration of medication by the hemodialysis system in response to changing patient glucose levels during a hemodialysis session. There is still a further need for automated and integrated glucose reading sensor and related alarms attendant to hemodialysis. There is need for monitoring and controlling glucose for a patient during an entire course of hemodialysis therapy. There is a need for automatically predicting possible alterations and incidences of hypoglycemia and hyperglycemia of the patient's glucose during a hemodialysis session.

### SUMMARY

The first aspect of the disclosure relates to a system. In any embodiment, the system can include a hemodialysis system comprising a control system, at least one medication infusion pump and a dialyzer fluidly connectable to a venous patient line and an arterial patient line and a glucose sensor in communication with the control system and positioned to continuously measure a glucose concentration of a patient during a hemodialysis treatment, the control system programmed to provide automated administration of medication by the at least one medication infusion pump in response to a change in the glucose concentration being outside of a specified range during the hemodialysis session.

In any embodiment, the glucose sensor can be positioned to measure a glucose concentration in the arterial patient line.

In any embodiment, the glucose sensor can be an infrared glucose sensor.

In any embodiment, the glucose sensor uses an electrode inserted into the arterial patient line.

In any embodiment, the glucose sensor can be an implantable glucose sensor.

In any embodiment, the glucose sensor can be positioned to measure a chemical composition of the skin of the patient to determine a glucose concentration.

In any embodiment, the glucose sensor can be a wearable glucose sensor.

In any embodiment, the glucose sensor uses Raman spectroscopy to measure the patient glucose level.

In any embodiment, the control system can be programmed to continuously provide the glucose concentration to a user.

In any embodiment, the system can include a monitor, wherein the control system can be programmed to provide the glucose concentration on the monitor.

In any embodiment, the control system can be programmed to provide an alert if the glucose concentration is outside of a specified range.

The features disclosed as being part of the first aspect of the disclosure can be in the first and third aspect of the disclosure, either alone or in combination.

The second aspect of the disclosure is drawn to a system. In any embodiment, the system can include a hemodialysis system comprising a control system, at least one medication infusion pump, and a dialyzer fluidly connectable to a venous patient line and an arterial patient line; and a glucose sensor positioned to continuously measure a glucose concentration in the arterial patient line, the control system programmed to communicate with the glucose sensor measuring the glucose level and to provide automated administration of medication by the at least one medication infusion pump in response to the glucose concentration being outside of a specified range.

In any embodiment, the control system can be programmed to provide the glucose concentration in the arterial patient line to a user.

In any embodiment, the system can include a monitor; the control system programmed to provide the glucose concentration in the arterial patient line on the monitor.

In any embodiment, the control system can be programmed to continuously provide the glucose concentration in the arterial patient line to a user.

In any embodiment, the control system can be programmed to provide automated administration of one of glucose or insulin by the at least one medication infusion pump in response to an incidence of hypoglycemia or hyperglycemia.

In any embodiment, the glucose sensor can be an infrared glucose sensor.

In any embodiment, the glucose sensor uses an electrode inserted into the arterial patient line.

In any embodiment, the control system can be programmed to provide an alert if the glucose concentration is outside of a specified range.

The features disclosed as being part of the second aspect of the disclosure can be in the first and third aspect of the disclosure, either alone or in combination.

The third aspect of the disclosure is drawn to a method. In any embodiment, the method can include the steps of providing a hemodialysis system comprising: a control system; at least one medication infusion pump; a glucose sensor in communication with the control system; and a dialyzer fluidly connectable to a venous patient line and an arterial patient line, continuously measuring a glucose concentration of a patient during a hemodialysis treatment, receiving via the control system a glucose concentration of a patient during a hemodialysis session; and automated administration of a medication by the at least one automated administration of a medication by the at least one medication infusion pump in response to the glucose concentration being outside of a specified range.

In any embodiment, the method can include receiving the glucose concentration in the arterial patient line of the hemodialysis system during the hemodialysis session.

In any embodiment, the method can include receiving the glucose concentration in a chemical composition of the skin of the patient during the hemodialysis session.

In any embodiment, the method can include displaying the glucose concentration on a monitor.

In any embodiment, the method can include providing an alert if the glucose concentration can be outside of a specified range.

The features disclosed as being part of the third aspect of the disclosure can be in the first and second aspect of the disclosure, either alone or in combination.

### BRIEF DESCRIPTION OF DRAWINGS

FIG. 1 is a schematic of a system including a glucose sensor for sensing glucose concentration of a patient during a hemodialysis session in accordance with any embodiments.

FIG. 2 is a block diagram of a system including a glucose sensor for sensing glucose concentration of a patient during a hemodialysis session in accordance with any embodiments.

FIG. 3 illustrates a system including a glucose sensor for sensing glucose concentration of a patient during a hemodialysis session in accordance with any embodiments.

FIG. 4 illustrates a system including a glucose sensor for sensing glucose concentration of a patient during a hemodialysis session in accordance with any embodiments.

FIG. 5 illustrates a system including a glucose sensor for sensing glucose concentration of a patient during a hemodialysis session in accordance with any embodiments.

FIG. 6 illustrates a system including a glucose sensor for sensing glucose concentration of a patient during a hemodialysis session in accordance with any embodiments.

FIG. 7 is a flow diagram illustrating a method in accordance with any embodiments described herein.

### DETAILED DESCRIPTION

Unless defined otherwise, all technical and scientific terms used generally have the same meaning as commonly understood by one of ordinary skill in the art.

The articles “a” and “an” are used to refer to one or to over one (i.e., to at least one) of the grammatical object of the article. For example, “an element” means one element or over one element.

The terms “administration,” administering,” “administer,” “delivering,” “deliver,” “introducing,” and “introduce” can be used, in context, interchangeably to indicate the introduction of a substance to a patient in need thereof, and can further mean the introduction of water, any agent or medication to a hemodialysis circuit where such water, agent or medication will enter the blood of the patient by diffusion, transversal of a diffusion membrane or other means.

The term “arterial line” refers to an intravenous line that is used for the withdrawal of blood from the patient for delivery to the hemodialysis machine.

The term “capacitive sensor” refers to a sensor that applies a voltage to an area and detects objects by measuring the changes in electric property (capacitance).

The term “Chronic kidney disease” (CKD) refers to a condition characterized by the slow loss of kidney function over time. The most common causes of CKD are high blood pressure, diabetes, heart disease, and diseases that cause inflammation in the kidneys. Chronic kidney disease can also be caused by infections or urinary blockages. If CKD progresses, end-stage renal disease (ESRD) can result, where the kidneys' function is inadequate to sustain life without supplemental treatment.

The terms “communicate” and “communication” include but are not limited to, the connection of system electrical elements, either directly or wirelessly, using optical, electromagnetic, electrical or mechanical connections, for data transmission among and between said elements.

The term “communication device” or “communication unit” refers to a device such as a telemetry system or any other alert system such as an audio feedback device, which can communicate monitoring results to a patient and/or a medical care personnel as needed. The term “communication device” in certain instances refers to a device which serves the purpose of sending information with transmission capabilities to another device which receives the information using receiving capabilities. The “communication device” can use electromagnetic, optical or acoustic means for signal transmission.

The terms “component” and “components” refer to a part or element of a larger set or system. As used herein, a component may be an individual element, or may be a grouping of components configured as a set, for example, as a cassette or a cleaning and/or disinfection manifold.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase the phrase “consisting of.” Thus, the phrase indicates that the limited elements are required or mandatory and that no other elements may be present.

The term “connectable” refers to being able to be joined together for purposes including but not limited to maintaining a position, allowing a flow of fluid, performing a measurement, transmitting power, and transmitting electrical signals. The term “connectable” can refer to being able to be joined together temporarily or permanently.

The term “continuously” refers to an act that occurs repeatedly or constantly, or without interruption or interference, during a given period.

The term “control signals” refers to energy that is provided from one element of a system to another element of a system to convey information from one element to another or to cause an action. For example, a control signal can energize a valve actuator to cause a valve to open or close. In another example a switch on a valve can convey the open or close state of a valve to a controller.

A “control system” consists of combinations of components that act together to maintain a system to a desired set of performance specifications. The performance specifications can include sensors and monitoring components, processors, memory and computer components configured to interoperate.

A “controller” or “control unit” is a device which monitors and affects the operational conditions of a given system. The operational conditions are typically referred to as output variables of the system, which can be affected by adjusting certain input variables.

The term “dialysate” describes a fluid into or out of which solutes from a fluid to be dialyzed diffuse through a membrane. A dialysate typically contains electrolytes that are close in concentration to the physiological concentration of electrolytes found in blood. A common sodium level for dialysate is approximately 140 mEq/L. Normal blood sodium levels can range from approximately 135 mEq/L to 145 mEq/L. The REDY system typically uses dialysate ranging from 120 mEq/L to 160 mEq/L. In certain embodiment, a “predetermined limit” or “predetermined concentration” of sodium values can be based off the common sodium levels for dialysate and normal blood sodium levels. “Normal” saline at 0/9% by weight and commonly used for priming dialyzers and extracorporeal circuits is 154 mEq/L.

The term “dialyzer” refers to a cartridge or container with two flow paths separated by semi-permeable membranes. One flow path is for blood and one flow path is for dialysate. The membranes can be in the form of hollow fibers, flat sheets, or spiral wound or other conventional forms known to those of skill in the art. Membranes can be selected from the following materials of polysulfone, polyethersulfone, poly(methyl methacrylate), modified cellulose, or other materials known to those skilled in the art.

The term “electrode” as used herein describes an electrical conductor used to make contact with a part of a fluid, a solid or solution. For example, electrical conductors can be used as electrodes to contact any fluid (e.g., dialysate) to measure the conductivity of the fluid or deliver or receive charge to the fluid. A “disc electrode” consists of an electrode with an electrode head in the shape of a disc. A “rod electrode” refers to an electrode in the shape of a rod or cylinder, with one end functioning as an electrode head. A “sheet electrode” refers to an electrode with an electrode head in the shape of a sheet. The sheet can be square, rectangular, circular or other solid planar geometries. A “mesh electrode” refers to an electrode with an electrode head consisting of a mesh, where a mesh is the same as that described for a mesh electrode. An “antenna electrode” refers to an electrode with an electrode head in the shape of an antenna, where antenna shape refers to a serpentine structure of conductive wires or strips. A “pin electrode” refers to a rod electrode with a small diameter. Other electrode and electrode head geometries can be considered.

The term “extracorporeal circuit” refers to a fluid pathway incorporating one or more components such as, but not limited to, conduits, valves, pumps, fluid connection ports or sensing devices configured therein such that the pathway conveys blood from a subject to an apparatus for hemodialysis, hemofiltration, hemodiafiltration or ultrafiltration and back to the subject.

The term “fluidly connectable” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

The term “glucose sensor” refers to a device which measures the glucose concentration in the blood. A blood glucose level of four grams, or about a teaspoon, is monitored for normal function in a number of tissues.

The term “hematocrit” refers to the ratio of the volume of red blood cells to the total volume of blood.

The term “hemodiafiltration” refers to a therapy that combines hemofiltration and hemodialysis.

The term “hemodialysis” refers to a technique where blood and a “cleansing fluid” called dialysate are exposed to each other separated by a semi-permeable membrane. Solutes within the permeability range of the membrane pass while diffusing along existing concentration gradients. Water and solutes are also transferred by convection across a pressure gradient that may exist across the hemodialysis membrane. The dialysate employed during hemodialysis has soluble ions such as sodium, calcium and potassium ions and is not pure water. The sieving properties of the membrane exclude certain solutes above a certain threshold from crossing the membrane. One common sieving property is “albumin sieving.” In certain embodiments, albumin may not be removed during renal replacement therapy because lower blood serum albumin is associated with increased mortality rates.

The terms “hemodialysis membrane,” “hemofiltration membrane,” “hemodiafiltration membrane,” “ultrafiltration membrane,” and generally “membrane,” refer, in context, to a semi-permeable barrier selective to allow diffusion and convection of solutes of a specific range of molecular weights through the barrier that separates blood and dialysate, or blood and filtrate, while allowing diffusive and/or convective transfer between the blood on one side of the membrane and the dialysate or filtrate circuit on the other side of the membrane.

The term “hemofiltration” refers to a therapy in which blood is filtered across a semi-permeable membrane. Water and solutes are removed from the blood via pressure-driven convection across the membrane. The sieving properties of the membrane exclude certain solutes above a certain threshold from crossing the membrane. One common sieving property is “albumin sieving.” In some situations, albumin may not be removed during renal replacement therapy because lower blood serum albumin can be associated with increased mortality rates. In hemofiltration, solutes small enough to pass through the membrane in proportion to their plasma concentration are removed. The driving force is a pressure gradient rather than a concentration gradient. A positive hydrostatic pressure drives water and solutes across the filter membrane from the blood compartment to the filtrate compartment where the filtrate can be drained. Solutes, both small and large, get dragged through the membrane at a similar rate by the flow of water that has been engineered by the hydrostatic pressure. Hence, convection overcomes the reduced removal rate of larger solutes (due to their slow speed of diffusion) observed in hemodialysis. The rate of solute removal is proportional to the amount of fluid removed from the blood circuit, which can be adjusted to meet the needs of a clinical situation. In general, the removal of large amounts of plasma water from the patient requires volume substitution. Substitution fluid, typically a buffered solution close to the plasma water composition a patient needs, can be administered pre or post filter (pre-dilution mode, post-dilution mode).

The term “in contact” as referred to herein denotes (a) a coming together or touching, as of objects or surfaces; or (b) the state or condition of touching or of being in immediate proximity. “In contact” also includes fluids that are “in fluid communication with” with a solid, such as for example, a fluid, like a dialysate, in contact with a material layer of a sorbent cartridge, or a fluid in contact with a sensor.

The term “infrared” refers to electromagnetic radiation having a wavelength from about 900 nm to 1 mm.

The term “infusion pump” refers to a device that can perform work on a fluid solution to cause the fluid flow and can actively control the transfer of fluid volume such as an infusate into a circuit.

The terms “introduce” and “introducing” refer to causing a substance to become present where the substance was not present, or to cause the amount or concentration of a substance to be increased.

The term “kidney disease” (KD) refers to a condition characterized by the slow loss of kidney function over time. The most common causes of KD are high blood pressure, diabetes, heart disease, and diseases that cause inflammation in the kidneys. Kidney disease can also be caused by infections or urinary blockages. If KD progresses, the disease can lead to end-stage renal disease (ESRD), where kidney function is inadequate to sustain life without supplemental treatment. KD can be referred to by different stages indicated by Stages 1 to 5. Stage of KD can be evaluated by glomerular filtration rate of the renal system. Stage 1 KD can be indicated by a GFR greater than 90 mL/min/1.73 m.sup.2 with the presence of pathological abnormalities or markers of kidney damage. Stage 2 KD can be indicated by a GFR from 60-89 mL/min/1.73 m.sup.2, Stage 3 KD can be indicated by a GFR from 30-59 mL/min/1.73 m.sup.2 and Stage 4 KD can be indicated by a GFR from 15-29 mL/min/1.73 m.sup.2. A GFR less than 15 mL/min/1.73 m.sup.2 indicates Stage 5 KD or ESRD. KD, as defined herein, contemplates KD regardless of the direction of the pathophysiological mechanisms causing KD and includes CRS Type II and Type IV and Stage 1 through Stage 5 KD among others. Kidney disease can further include acute renal failure, acute kidney injury, and worsening of renal function.

The terms “measuring,” to “measure,” or “measurement” determining a state or parameter of a system or substance.

The term “memory” refers to any device for recording digital information that can be accessed by a microprocessor, such as RAM, Dynamic RAM, microprocessor cache, FLASH memory, or memory card.

The term “patient” or “subject” refers to a member of any animal species, preferably a mammalian species, optionally a human. The subject can be an apparently healthy individual, an individual suffering from a disease, or an individual being treated for an acute condition or a chronic disease

The term “peritoneal hemodialysis” refers to a therapy wherein a dialysate is infused into the peritoneal cavity, which serves as a natural dialyzer. In general, waste components diffuse from a patient's bloodstream across a peritoneal membrane into the hemodialysis solution via a concentration gradient. In general, excess fluid in the form of plasma water flows from a patient's bloodstream across a peritoneal membrane into the hemodialysis solution via an osmotic gradient.

The term “position” or “positioned” refers to a physical location of a component or system.

The terms “processor,” “computer processor,” and “microprocessor” as used herein are broad terms and are to be given their ordinary and customary meaning to a person of ordinary skill in the art. The terms refer without limitation to a computer system, state machine, processor, or the like designed to perform arithmetic or logic operations using logic circuitry that responds to and processes the basic instructions that drive a computer. In any embodiment, the terms can include ROM (“read-only memory”) and/or RAM (“random-access memory”) associated therewith.

The term “programmable,” “programmed,” and the like as used herein refers to a device using computer hardware architecture and being capable of carrying out a set of commands, automatically.

The term “pump” refers to any device that causes the movement of fluids or gases by the application of suction or pressure.

The term “Raman spectroscopy” refers to a molecular spectroscopic technique based on a light scatting process wherein light interacts with matter to provide a material's make up or characteristics.

The term “sensor,” which can also be referred to as a “detector” in certain instances, and as used herein can be any or combination of a mechanical transducer, converter, optical, electrical, or any other sensing modality that can detect, measure, or sense a physical or otherwise measurable quantity of a matter in a solution, liquid, gas, or combinations thereof. In any non-limiting embodiment, the sensor can measure chemicals, solute concentration, flow, density, content, or any other measurand and convert the obtained value into a signal of any type which can be read by an instrument, circuit, computer, processor, or any other suitable component or device.

The term “sensor element” refers to a device or component of a system that detects or measures a physical property.

The term “spectroscopy” refers to the study of light as a function of the wavelength emitted or reflected through a solid, liquid or gas.

The term “spectrophotometry sensor” refers to a sensor that uses photometers to measure the intensity of a light beam at different wavelengths.

The terms “treating” and “treatment” refer to the management and care of a patient having a pathology or condition by administration of one or more therapy contemplated herein. Treating also includes administering one or more methods as described herein or using any of the systems, devices or compositions as described herein in the treatment of a patient. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

The terms “treatment” or “therapy” refer to both therapeutic treatment and prophylactic or preventative measures. “Treating” or “treatment” does not require complete alleviation of signs or symptoms, does not require a cure, and includes protocols having only a marginal or incomplete effect on a patient.

The term “venous line” refers to an intravenous line for the input of medicine or nutrition into the bloodstream.

### Hemodialysis System Including Continuous Glucose Monitoring

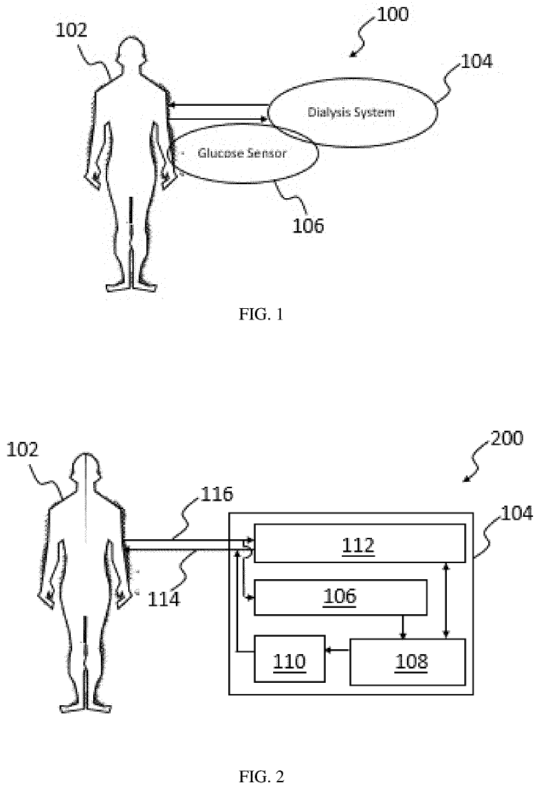

FIG. 1 shows a patient monitoring system operating in a continuous manner during a hemodialysis session in which blood glucose levels are monitored and medications may be administered automatically in response to blood glucose levels outside a desired range. In a hemodialysis session, a patient's blood is dialyzed against a dialysate through an artificial hemodialysis membrane. One goal during hemodialysis, hemodiafiltration, hemofiltration, ultrafiltration, and like treatments is to ensure that the patient's blood glucose concentration is within an acceptable range. In any embodiment, the hemodialysis system and methods can identify out-of-range patient glucose concentrations. Typically, a patient's blood glucose level can range during or following a blood fluid removal session from about 100 mg/dL (5.55 mmol/L) to about less than 140 mg/dL (7.8 mmol/L). During a hemodialysis session, glucose levels can sometimes dip further to about (<5.55 mmol/L). Such low glucose levels can be associated with increased mortality risk. Conditions that may affect a patient's blood glucose level during a hemodialysis include nutritional deficiencies, reduced ability of the kidneys to form new glucose molecules, loss of glucose to the dialysate, decreased insulin clearance, and glucose diffusion into erythrocytes. These conditions may present during a hemodialysis session as changes in glucose metabolism, insulin resistance/secretion/degradation and changes in drug metabolism to treat such.

The methods, systems and devices described herein may be used, in any embodiment, to determine an initial blood glucose level based on monitoring that occurs before a blood fluid removal or hemodialysis session starts. The hemodialysis session can also be referred to as a blood fluid removal session wherein the blood glucose level can change at any point during the hemodialysis session. In any embodiment, the systems and methods can monitor the patient's glucose levels intermittently, periodically or continuously over the course of the hemodialysis session. To minimize interference with the patient, the blood glucose sensing component(s) thereof, can be built into the hemodialysis system, or be implanted or configured as a wearable. Suitable glucose sensing components can be described to automatically detect and monitor glucose levels.

In any embodiment, the systems and methods can provide medication to the patient during hemodialysis in response to sensed changes in blood glucose levels outside of a specified range. The delivery of medication can be automated to occur upon receiving certain specified glucose values or other monitored physiological measurements that recommend the delivery of a particular medicament.

In any embodiment, one or more sensors can be employed to detect glucose levels in the body of the patient. The glucose levels can be detected in blood in vivo, a blood sample, on skin surface contact, via a measurement in interstitial tissue, or any other body compartment of combination of body compartments that can provide a measured or predicted value of glucose in the patient. For example, a blood glucose sensor that measures glucose concentration by direct contact with bodily fluids can be employed. Alternatively, a blood glucose sensor that measure glucose concentration by indirect contact with bodily fluids, such as through infrared glucose measurement on an arterial blood line can be employed. Similarly, external or implantable glucose sensors can be used to measure the chemical composition of the skin to determine the amount of glucose and can optionally be used in conjunction with a sensor that takes measurements through direct contact with bodily fluids if necessitated. In any embodiment, the sensor can be replaced subsequent to its useful life. The output from any one or more combination of sensors of different types can be used together to develop a glucose level or glucose status of a patient. In any embodiment, more than one sensor can be employed for purposes of result confirmation and redundancy, which can improve reliability and accuracy. In any embodiment, the blood glucose sensor may be configured to accurately detect different ranges of glucose concentration.

FIG. 1 is a schematic showing an overview of a system 100 for sensing blood glucose and providing automated medication such as through the infusion of glucose and/or insulin, to a patient during a hemodialysis session. The system 100 includes a hemodialysis system 104 and a glucose sensor 106 in communication with the hemodialysis system 104. The glucose sensor 106 is positioned to continuously measure a glucose concentration of a patient 102 during a hemodialysis treatment. The hemodialysis system 104 is configured to provide automated administration of a medication in response to changing patient glucose levels outside of a desired range during the hemodialysis session.

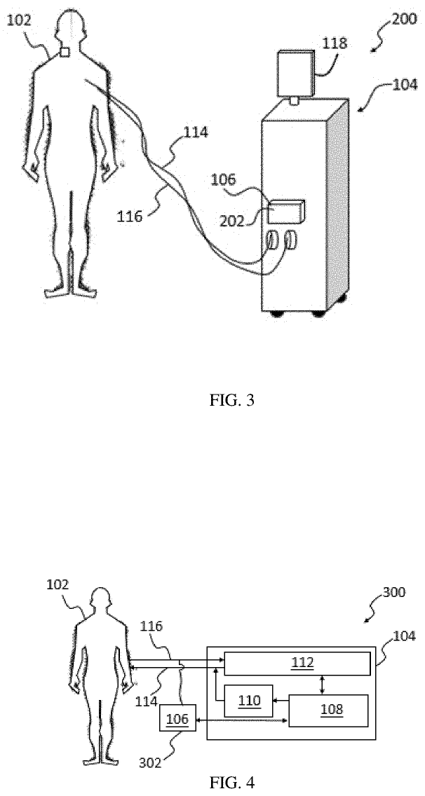

In FIG. 2 and FIG. 3, a system 200 includes the hemodialysis system 104 comprising a control system 108, at least one medication infusion pump 110, and a dialyzer 112 fluidly connectable to a venous patient line 114 and an arterial patient line 116. The system 200 further includes the glucose sensor 106 in communication with the control system 108. The glucose sensor 106 is positioned to continuously measure a glucose concentration of the patient 102 during a hemodialysis treatment. The control system 108 is programmed to provide automated administration of at least one medication by the at least one medication infusion pump 110 in response to changing patient glucose levels during the hemodialysis session that fall outside of a desired range. One of ordinary skill will understand that features of the systems disclosed herein that have been previously described with regard to the schematic of FIG. 1 will be referenced throughout the figures as having the same reference number.

In FIG. 2 and FIG. 3, the glucose sensor 106 and at least one medication infusion pump 110 can be an integral part of the hemodialysis system 104. In any embodiment, the glucose sensor 106 can be optically coupled to the arterial patient line 116. More particularly, the glucose sensor 106 can utilize spectroscopy, such as infrared spectroscopy, to perform continuous non-invasive measurements of the blood glucose level of the patient on a continual basis during the hemodialysis session. In any embodiment, the glucose sensor 106 (which may include an associated light source(s)) may be placed in any suitable location, such as around the tubing that carries blood from the patient to the blood fluid removal device or from the blood fluid removal device to the patient.

In any embodiment, the hemodialysis system 104 as shown in FIG. 3 can incorporate a multi-therapeutic approach to extracorporeal blood treatment. In any embodiment, the hemodialysis system 104 can perform hemodiafiltration with endogenous reinfusion therapy to combine the benefits of a dual chamber dialyzer with a specific adsorbent cartridge. This combination can improve selective clearance and biocompatibility for patients suffering from more serious malnutrition and inflammatory conditions. In any embodiment, the hemodialysis system 104 can perform mid-dilution hemodiafiltration to combines post and pre-dilution to administer an infusion directly in the middle of a specially designed two-stage high flux dialyzer. In any embodiment, the hemodialysis system 104 can perform on-line hemodialysis in any infusion modes such as pre, post, and pre+post dilution. In any embodiment, the hemodialysis system 104 can perform on-line hemodiafiltration during an isolated ultrafiltration mode during on-line hemodiafiltration and mid-dilution hemodiafiltration to take advantage of the benefits of pure convection. Low flux and high flux standard hemodialysis can be provided in a double-needle or single-needle mode, with one or two pumps.

In any embodiment, the hemodialysis system 104 can be configured to monitor any one more of a hematocrit, oxygen saturation, blood glucose level, temperature, and combinations thereof. In any embodiment, the hemodialysis system 104 can be a hemodialysis system including a plurality of built-in sensors such as the Medtronic Flexya. The hemodialysis system 104 can provide a multitherapeutic approach to extracorporeal blood treatment with personalized therapy. The hemodialysis system 104 can have any number of pumps and include software to perform any number of hemodialysis treatments to meet the therapeutic needs of a particular patient. The hemodialysis system 104 can include additional sensors (not shown) for real-time monitoring of dialysis progress and patient status in biofeedback to help prevent any complication.

In any embodiment, the hemodialysis system 104 can provide real-time monitoring of hemodialysis progress and patient status via multiple sensors, one of which can include the glucose sensor 106. In any embodiment, the glucose sensor 106 can be configured to use a dual wavelength spectrophotometry sensor 202 providing for the measurement of the optical properties of the blood as indicative of blood glucose levels. The resulting signal can be fed to the control system 108, which can provide the obtained data on a monitor 118. In response to obtained blood glucose data outside of a specified range, the hemodialysis system 104, and more particularly, the at last one medication infusion pump 110 in response to a signal indicating blood glucose levels being outside a specified range, receives a signal to automatically infuse a medication into the recirculating blood flow path, such as into the patient line 114, to bring the blood glucose levels back into the specified range.

In any embodiment, the hemodialysis system 104 can respond to patient diversity and clinical complexity by monitoring biofeedback systems to recognize patient situations based on biochemical signals. The hemodialysis system 104 can monitor and recognize clinically significant conditions monitored during a dialysis session. In any embodiment, the hemodialysis system 104 can check for intra-dialysis complications. In any embodiment, the hemodialysis system 104 can check a patient's body temperature at a preset value according to medical prescription (isothermal or hypothermal method). In any embodiment, the hemodialysis system 104 can control dialysis treatment, reducing the risk of possible hypotensive events for the patient. In any embodiment, the hemodialysis system 104 can modulate ultrafiltration and dialysate conductivity according to personalized profiles. By simulating the intradialytic kinetics of the solute and fluid exchanges, the hemodialysis system 104 can control the plasma sodium concentration and maintains the osmolar equilibrium, to prevent primary and post dialysis clinical complications, such as hypotension and disequilibrium syndrome. In any embodiment, the hemodialysis system 104 can perform real-time monitoring of oxygen saturation to provide information on the status of peripheral circulation. If a “safety level” value is exceeded, the hemodialysis system 104 can generate an alert as to the possible onset of a hypotensive event.

In FIG. 4, a system 300 includes the hemodialysis system 104 having a control system 108, at least one medication infusion pump 110, and a dialyzer 112 fluidly connectable to a venous patient line 114 and an arterial patient line 116. The system 300 further includes the glucose sensor 106 in communication with the control system 108. The glucose sensor 106 can be positioned to continuously measure a glucose concentration of the patient 102 during a hemodialysis treatment. The control system 108 is programmed to provide automated administration of at least one medication by the at least one medication infusion pump 110 in response to changing patient glucose levels during the hemodialysis session. Notably, the glucose sensor 106 can be disposed separate and apart from the hemodialysis system 104. As illustrated in FIG. 4, the glucose sensor 106 can be in communication with the control system 108 via a wired or wireless coupling and the arterial patient line 116 to provide continuous monitoring of the blood glucose level of a patient 102 during a hemodialysis session.

In the embodiment of FIG. 4, the glucose sensor 106 can be a capacitive sensor 302 in which an electrode is inserted in the arterial patient line 116 to obtain blood glucose data. Alternatively, the glucose sensor 106 may be configured to include an electrode that is suitable location relative to patient 102 to measure the glucose values. In any embodiment, the at least one medication infusion pump 110 can be configured as an integral part of the hemodialysis system 104. In this particular embodiment, the glucose sensor 106 can be coupled to the arterial patient line 116 and to the control system 108 via a wired or wireless coupling. The glucose sensor 106 can be configured to perform continuous non-invasive measurements of the blood glucose level of the patient on a continual basis during the hemodialysis session. During monitoring, the glucose sensor 106 obtains a blood glucose level measurement and a resulting signal can be fed to the control system 108, which may or may not provide the obtained data on a monitor. In response to obtained blood glucose data outside of a specified range, the hemodialysis system 104, and more particularly, the at last one medication infusion pump 110 in response to a signal indicating blood glucose levels being outside a specified range, receives a signal to automatically infuse a medication to bring the blood glucose levels back into the specified range.

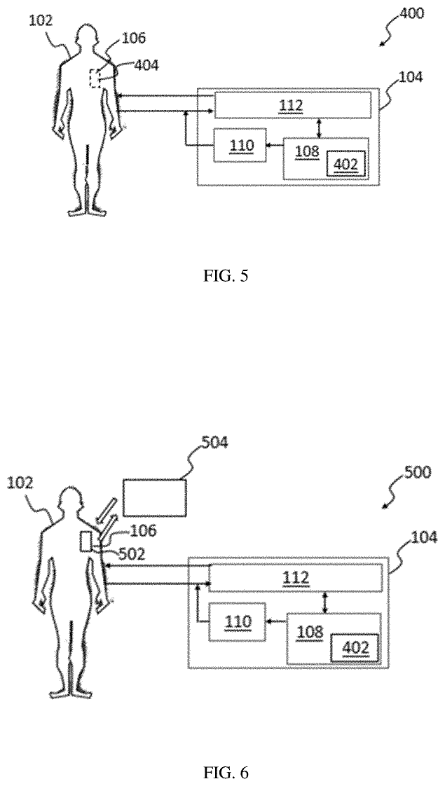

Referring now to FIG. 5, a system 400 includes the hemodialysis system 104 comprising a control system 108, at least one medication infusion pump 110, and a dialyzer 112 fluidly connectable to a venous patient line 114 and an arterial patient line 116. The system 400 further includes a glucose sensor 106 in communication with the control system 108. The glucose sensor 106 can be positioned to continuously measure a glucose concentration of the patient 102 during a hemodialysis treatment. The control system 108 can be programmed to provide automated administration of at least one medication by the at least one medication infusion pump 110 in response to changing patient glucose levels during the hemodialysis session. Similar to the embodiment of FIG. 4, the glucose sensor 106 is disposed separate and apart from the hemodialysis system 104. As illustrated, the glucose sensor 106 can be in communication with the control system 108 via a glucose sensor receiver 402 to provide continuous monitoring of the blood glucose level of a patient 102 during a hemodialysis session.

In FIG. 5, the glucose sensor 106 can be an implantable blood glucose sensor 404, that can be implanted subcutaneously under the patient's skin to obtain blood glucose data. Alternatively, the glucose sensor 106 can be a partially implantable blood glucose sensor 404 wherein the glucose sensor 106 is configured to include an electrode that is inserted just under the skin of the patient 102 to measure the glucose values. In any embodiment, the glucose sensor 106 can be the Medtronic ENLITE device. Such electrodes, and components of sensors employing such electrodes, are known in the art and may be employed, or modified to be employed, for use in the monitoring described herein. Generally similar to the previous embodiment, in the embodiment of FIG. 5, the at least one medication infusion pump 110 can be configured as an integral part of the hemodialysis system 104. In any embodiment, the infusion pump 110 can be integral or work in conjunction with a link transmitter, e.g., Medtronic Guardian™ 2, wherein glucose information is sent directly to an insulin pump, e.g., MiniMed™ insulin pump. For example, in an embodiment, a sensor would be placed on the patient once a week and connected wirelessly to a MiniMed™ pump during each dialysis session. A typical sensor has a useful life of six (6) days and would be replaced at the end of this time with a new sensor. In another embodiment, a luer-lock style connector could be used to fit the venous or arterial line connections and infuse the medication through the venous or arterial line. In any embodiment, the infusion pump 110, the glucose sensor 106 can be compatible or paired to otherwise work in concert with any suitable smart pump technology systems during dialysis therapy.

In any embodiment, the glucose sensor 106 can be coupled to the control system 108 via a wireless coupling. In an alternate embodiment, the glucose sensor 106 can be coupled to the control system 108 via a wired coupling. The glucose sensor 106 can be configured to perform measurements of the blood glucose level of the patient on a continual basis during the hemodialysis session. During monitoring, the glucose sensor 106 obtains a blood glucose level measurement and a resulting signal is fed to the control system 108, which may or may not provide the obtained data on a monitor or to a user. In response to obtained blood glucose data outside of a specified range, the hemodialysis system 104, and more particularly, the at last one medication infusion pump 110 in response to a signal indicating blood glucose levels being outside the specified range, receives a signal to automatically infuse the medication to bring the blood glucose levels back into the specified range.

In FIG. 6, a system 500 including the hemodialysis system 104 comprises a control system 108, at least one medication infusion pump 110, and a dialyzer 112 fluidly connectable to a venous patient line 114 and an arterial patient line 116. The system 500 further includes the glucose sensor 106 in communication with the control system 108. The glucose sensor 106 can be positioned to continuously measure a glucose concentration of the patient 102 during a hemodialysis treatment. The control system 108 is programmed to provide automated administration of at least one medication by the at least one medication infusion pump 110 in response to changing patient glucose levels during the hemodialysis session. Similar to the embodiments of FIG. 4 and FIG. 5, the glucose sensor 106 can be disposed separate and apart from the hemodialysis system 104. As illustrated, the glucose sensor 106 can be in communication with the control system 108 via a glucose sensor receiver 402 to provide continuous monitoring of the blood glucose level of a patient 102 during a hemodialysis session.

In the embodiment of FIG. 6, the blood glucose sensor 106 can be a wearable blood glucose sensor 502, that is in contact with the patient's skin to obtain blood glucose data. More particularly, the glucose sensor 106 can be a non-invasive, wireless, continuous glucose monitoring system. In any embodiment, the blood glucose sensor 502 can utilize Raman spectroscopy 504 to measure the chemical composition of the patient's skin to determine the level of blood glucose. The measurement would be performed on the patient's skin in a non-invasive manner Generally similar to the previous embodiment, in the embodiment of FIG. 6, the at least one medication infusion pump 110 is configured as an integral part of the hemodialysis system 104. In this particular embodiment, the glucose sensor 106 is coupled to the control system 108 via a wireless coupling. In an alternate embodiment, the glucose sensor 106 is coupled to the control system 108 via a wired coupling. The glucose sensor 106 is configured to perform measurements of the blood glucose level of the patient on a continual basis during the hemodialysis session. During monitoring, the glucose sensor 106 obtains a blood glucose level measurement and a resulting signal is fed to the control system 108, which may or may not provide the obtained data on a monitor or to a user. In response to obtained blood glucose data outside of a specified range, the hemodialysis system 104, and more particularly, the at last one medication infusion pump 110 in response to a signal indicating blood glucose levels being outside a specified range, receives a signal to automatically infuse the medication to bring the blood glucose levels back into the specified range.

In any of the systems previously described, the user can continuously monitor a patient's glucose values on the hemodialysis monitor screen, schedule alarms with desired maximum and minimum values, and activate acoustic warnings. The system disclosed herein continuously monitors certain parameters (in this case the glucose of a diabetic patient on hemodialysis). The system performs a continuous measurement of glucose throughout the hemodialysis treatment, these measurements are displayed in the hemodialysis monitor interface, indicating the initial glucose values of the patient and the constant values during treatment, can also offer an evolution graph throughout the hemodialysis session. The display has user configurable parameters with maximum and minimum alarm limits with visual and acoustic alarms. In this way the user can anticipate an incident of hyperglycemia or hypoglycemia during hemodialysis treatment, thus achieving more hemodynamic stability and treatment.

In any of the systems previously described, the system provides the automatic administration of medication if the patient's glucose values exceed configured safety parameters ranges. To provide for the automatic administration of medication, the system can be preprogrammed with desired parameters, such as g/dl of glucose to be administered, an alarm parameter to start glucose administration, program the insulin units to be administered when the patient's values exceed the programmed maximum glycemic range parameter, etc.

Any of the glucose sensors 106 of the system 100 disclosed herein may be calibrated prior to sensing (in any condition mimicking the final sensor environment) with a known glucose concentration. The sensors can be recalibrated subsequent to placement relative to the patient. For example, blood glucose level can be measured external to the patient, e.g., via blood draws, and results of the external monitoring can be communicated to the sensor by receiving input, e.g., from healthcare providers. Thus, the sensor can recalibrate based on the input regarding the external measurements if necessitated. Alternatively, or in addition, the sensor may have an internal reference built in. In cases where the sensor outputs raw data to an external device, the external device may be calibrated to interpret the raw data from the sensor with regard to input regarding the external measurements.

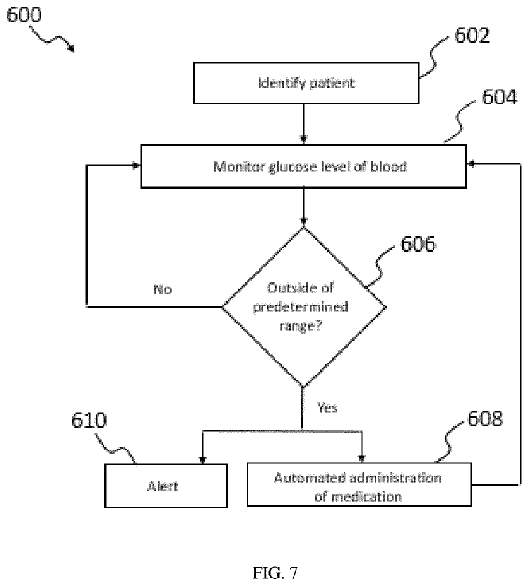

Referring now to FIG. 7, a depicted method 600 includes steps for identifying, selecting or diagnosing a patient for which a blood fluid removal or hemodialysis session is indicated in a step 602 and monitoring blood glucose levels of the blood of the patient in s a step 604. The monitoring step 604 may be continuous and may employ one or more glucose sensors as previously described. The method 600 in FIG. 7 can include determining whether the blood glucose level is out of range in step 606 based on data acquired during the monitoring step 604. For example, a determination step 606 can be made as to whether the blood glucose level crossed a threshold (e.g., a ceiling or floor). Suitable thresholds or ranges may be stored in, for example, a look-up table in memory of a sensor device, the blood fluid removal device, or other suitable device for purposes of determining whether the blood glucose level is out of range in a step 606 based on data acquired during the monitoring. If the blood glucose level is determined to be within range, monitoring step 604 may continue. If the blood glucose level is determined to be out of range (e.g., cross a threshold), automated administration of medication to bring the blood glucose levels back into the specified range is performed in step 608. Subsequent to the administration of the medication, monitoring step 604 can include determining whether blood glucose levels are in a specified range.

In addition, as a result of the monitoring step 604, when the blood glucose level is determined to be out of range, any suitable alert may be issued in step 610. The alert may be a tactile cue, such as vibration or audible alarm, generated by a sensor or a device in communication with the sensor, such as the hemodialysis system. The alert may provide the patient and/or healthcare provider with notice that medical attention should be sought. The alert may also provide information to the healthcare provider regarding the nature of the health issue (e.g., blood glucose level out of range) and treatment (e.g., blood fluid removal session) for which the alert in step 610 was issued. The sensor or the device in communication with the sensor may alert the healthcare provider by transmitting the alert or related information over the internet, a telephone network, via a monitor, or other suitable network or component to a device in communication with the healthcare provider.

### EXAMPLE

Diabetes patient Mellitus Type 2

### 68 Years

4 hours of hemodialysis. QB: 350 ml/h. UF/Total:2.5 Kg. Initial Arterial Pressure 122/76 mmHG A/V: FAVI Initial Glucose: 120 mg/dl
The patient suffers from hypoglycemia within 3 hours of treatment, sweating, cold and moist skin, slight paleness, feeling dizzy, tachycardia, restlessness and discreet hypertension. The patient's glucose measurement is performed and has a glucose of 73 mg/dL.
With a continuous glucose sensor during the hemodialysis session, this incident would have been detected in advance, since being able to visualize the data on screen constantly throughout the duration of treatment, a thorough monitoring and monitoring of the patient is obtained, ensuring the stability of the same during treatment. In addition, by being able to configure alarm parameters, variation in parameters can be anticipated, improving the comorbidity of the renal patient. An option to administer glucose or insulin through the medication infusion pumps of the hemodialysis system provides more effective patient care and reaction of healthcare personnel when acting on such an incident.

As indicated above, sensors for monitoring patient physiological parameters may be, or may have components that are, contained within the hemodialysis system, implantable or wearable. In embodiments, multiple sensors may be connected via telemetry, body bus, or the like. The connected sensors may be of the same or different type. Such connected sensors may be placed (e.g., internal or external) for purposes of monitoring at various locations of the patient's body.

Monitoring may alternatively or additionally include receiving patient or physician feedback regarding the patient's state. For example, the patient may indicate a point in time when nausea begins, which often happens when blood glucose levels are too high. The blood fluid monitoring device may include an input, such as a keyboard or touch screen display for entering such data. Alternatively, a separate device such as a patient programmer, laptop computer, tablet computer, personal data assistance, smart phone or the like may be used to input the data; or the like.

The disclosed system for monitoring blood glucose levels of a patient during a hemodialysis system and the automatic administration of medication during the session to address blood glucose levels outside of a specified range allows the diabetic patient to be monitored throughout the hemodialysis session, ensuring patient stability and allows for predicting possible alterations and incidences (hypoglycemia, hyperglycemia) of the patient during the hemodialysis session.

The particular embodiments disclosed above are illustrative only, and may be modified and practiced in different but equivalent manners apparent to those skilled in the art having the benefit of the teaching provided herein. Furthermore, no limitations are intended with respect to the details of construction, or the design shown herein, other than as described in the claims below. One skilled in the art will understand that various combinations and/or modifications and variations can be made in the systems and methods depending upon the specific needs for operation. Features illustrated or described as being part of an aspect as described herein can be used in any other aspect as described herein, either alone or in combination.

1. A system, comprising:
a hemodialysis system; the hemodialysis system comprising a control system, at least one medication infusion pump, and a dialyzer fluidly connectable to a venous patient line and an arterial patient line; and
a glucose sensor in communication with the control system; the glucose sensor positioned to continuously measure a glucose concentration of a patient during a hemodialysis treatment,
the control system programmed to provide automated administration of medication by the at least one medication infusion pump in response to a change in the glucose concentration being outside of a specified range during the hemodialysis session.

2. The system of claim 1, the glucose sensor positioned to measure a glucose concentration in the arterial patient line.

3. The system of claim 2, wherein the glucose sensor is an infrared glucose sensor.

4. The system of claim 2, wherein the glucose sensor uses an electrode inserted into the arterial patient line.

5. The system of claim 1, wherein the glucose sensor is an implantable glucose sensor.

6. The system of claim 1, the glucose sensor positioned to measure a chemical composition of the skin of the patient to determine a glucose concentration.

7. The system of claim 1, wherein the glucose sensor is a wearable glucose sensor.

8. The system of claim 7, wherein the glucose sensor uses Raman spectroscopy to measure the patient glucose level.

9. The system of claim 1, the control system programmed to continuously provide the glucose concentration to a user.

10. The system of claim 1, further comprising a monitor; the control system programmed to provide the glucose concentration on the monitor.

11. The system of claim 1, the control system programmed to provide an alert if the glucose concentration is outside of a specified range.

12. A system, comprising:
a hemodialysis system; the hemodialysis system comprising a control system, at least one medication infusion pump, and a dialyzer fluidly connectable to a venous patient line and an arterial patient line; and
a glucose sensor; the glucose sensor positioned to continuously measure a glucose concentration in the arterial patient line,
the control system programmed to communicate with the glucose sensor measuring the glucose level and to provide automated administration of medication by the at least one medication infusion pump in response to the glucose concentration being outside of a specified range.

13. The system of claim 12, the control system further comprising a monitor, the control system further programmed to provide the glucose concentration in the arterial patient line to at least one of a user and the monitor.

14. The system of claim 12, the control system programmed to continuously provide the glucose concentration in the arterial patient line to a user.

15. The system of claim 12, the control system programmed to provide automated administration of one of glucose or insulin by the at least one medication infusion pump in response to an incidence of hypoglycemia or hyperglycemia.

16. The system of claim 12, wherein the glucose sensor is an infrared glucose sensor.

17. The system of claim 12, wherein the glucose sensor uses an electrode inserted into the arterial patient line.

18. The system of claim 12, the control system programmed to provide an alert if the glucose concentration is outside of a specified range.

19. A method, comprising the steps of:
providing a hemodialysis system comprising:
a control system;
at least one medication infusion pump;
a glucose sensor in communication with the control system; and
a dialyzer fluidly connectable to a venous patient line and an arterial patient line,
continuously measuring a glucose concentration of a patient during a hemodialysis session;
receiving via the control system the glucose concentration of the patient during the hemodialysis session; and
automated administration of a medication by the at least one medication infusion pump in response to the glucose concentration being outside of a specified range.

20. The method of claim 19, further comprising receiving the glucose concentration in the arterial patient line of the hemodialysis system during the hemodialysis session.

21-23. (canceled)
